(19) United States
(12) Patent Application Publication
(10) Pub. No.: US 2021/0212883 A1
(43) Pub. Date: Jul. 15, 2021

(54) THERAPEUTIC ROLLER WITH CONDUCTIVE LAYER

(71) Applicant: MedRock, Inc., Portland, OR (US)

(72) Inventors: Tony ROCKLIN, Portland, OR (US); Jeffrey Daniel ALLISON, Lake Oswego, OR (US)

(73) Assignee: MedRock, Inc., Portland, OR (US)

(21) Appl. No.: 16/743,381

(22) Filed: Jan. 15, 2020

(51) Int. Cl. A61H 15/00; A61H 23/02

(52) U.S. Cl. CPC A61H 15/00; A61H 2201/0228; A61H 2015/0014; A61H 23/02

(57) ABSTRACT

A thermally active roller apparatus, which includes a core; a compliant layer disposed about the core; a cover disposed about the compliant layer; a heating element; and a thermally conductive layer disposed around the heating element. Included is a thermally active roller apparatus in which the compliant layer may be formed from two or more portions having different stiffnesses. A thermally active or non-thermally active roller apparatus that includes a compliant layer comprising at least two portions of different stiffnesses and wherein the at least two portions are interdigitated to lock them together.

### FIELD

Embodiments herein relate to tools for preparation and recovery for athletic activity as well as treatment of muscle and/or joint dysfunctions. Specifically, embodiments herein relate to tools for self-massage and/or myofascial release.

### BACKGROUND

Self-applied muscle and joint therapy products may be used by patients and athletes to:
1. Increase circulation to get more oxygen and nutrients to soft tissues and vital organs;
2. Enhance muscle relaxation;
3. Optimize correction of muscle imbalances;
4. Improve joint range of motion;
5. Decrease pain;
6. Decrease muscle spasms;
7. Use as a warm-up for tissues prior to work-out or activity;
8. Use as a post-work out tool to push lactic acid out of tissues and reduce recovery time;
9. Stimulate the flow of lymph, the body's natural defense system, against toxic invaders;
10. Release endorphins, the body's natural painkiller, to control and relieve pain for chronic illness, injury and recovery from surgery;
11. Reduce post-surgery adhesions and edema while also being used to reduce and realign scar tissue after healing has occurred;
12. Improve neuromuscular efficiency;
13. Reduce trigger point sensitivity;
14. Optimize length-tension relationships throughout the human body which reduces stress on movement patterns.

One category of self-applied therapy devices may be commonly referred to as “foam rollers.” Foam rollers may come in many shapes and sizes but generally make use of the user's own mass to enable targeted pressure application to specific areas on the body.

### DESCRIPTION OF DRAWINGS

FIG. 1 depicts an isometric view of a heated therapeutic roller with integrated heating apparatus, in accordance with various embodiments.

FIG. 2 depicts an isometric cross-sectional view of a heated therapeutic roller with comfort foam and heating elements, in accordance with various embodiments.

FIG. 3 depicts an exploded view of a heated therapeutic roller with comfort foam and heating elements, in accordance with various embodiments.

FIG. 4 depicts various layers of a therapeutic roller with comfort foam and heating elements, in accordance with various embodiments.

FIG. 5 depicts a heated therapeutic roller with AC power, in accordance with various embodiments.

FIG. 6 depicts a heated therapeutic roller with DC power, in accordance with various embodiments.

FIG. 7 depicts a heated therapeutic roller with vibration added, in accordance with various embodiments.

FIG. 8 depicts a heated therapeutic roller with a thermal reservoir, in accordance with various embodiments.

FIG. 9 depicts a heated therapeutic roller sleeve, in accordance with various embodiments.

FIGS. 10 and 11 depict a heated therapeutic roller comfort foam layer, in accordance with various embodiments.

FIG. 12 depicts an isometric cross-sectional view of a heated therapeutic roller with comfort foam and heating elements, in accordance with various embodiments.

FIG. 13 depicts an endcap of a heated therapeutic roller, in accordance with various embodiments.

FIG. 14 depicts a therapeutic roller having a duel density surface, in accordance with various embodiments.

FIG. 15 depicts a therapeutic roller having a duel density surface, in accordance with various embodiments.

### DESCRIPTION

Various aspects of the illustrative embodiments will be described using terms commonly employed by those skilled in the art to convey the substance of their work to others skilled in the art. However, it will be apparent to those skilled in the art that alternate embodiments may be practiced with only some of the described aspects. For purposes of explanation, specific devices and configurations are set forth in order to provide a thorough understanding of the illustrative embodiments. However, it will be apparent to one skilled in the art that alternate embodiments may be practiced without the specific details. In other instances, well-known features are omitted or simplified in order not to obscure the illustrative embodiments.

Further, various operations will be described as multiple discrete operations, in turn, in a manner that is most helpful in understanding the present disclosure. However, the order of description should not be construed as to imply that these operations are necessarily order dependent. In particular, these operations need not be performed in the order of presentation.

The phrase “in an embodiment” is used repeatedly. The phrase generally does not refer to the same embodiment; however, it may. The terms “comprising,” “having,” and “including” are synonymous, unless the context dictates otherwise.

In providing some clarifying context to language that may be used in connection with various embodiments, the phrases “A/B” and “A and/or B” mean (A), (B), or (A and B); and the phrase “A, B, and/or C” means (A), (B), (C), (A and B), (A and C), (B and C) or (A, B and C).

The term “coupled with,” along with its derivatives, may be used herein. “Coupled” may mean one or more of the following. “Coupled” may mean that two or more elements are in direct physical or electrical contact. However, “coupled” may also mean that two or more elements indirectly contact each other, but yet still cooperate or interact with each other, and may mean that one or more other elements are coupled or connected between the elements that are said to be coupled with each other.

As noted above, self-applied muscle and joint therapy products such as foam rollers may be used by any individual, including patients and athletes, to:
1. Increase circulation to get more oxygen and nutrients to soft tissues and vital organs;
2. Enhance muscle relaxation;
3. Optimize correction of muscle imbalances;
4. Improve joint range of motion;
5. Decrease pain;
6. Decrease muscle spasms;
7. Use as a warm-up for tissues prior to work-out or activity;
8. Use as a post-work out tool to push lactic acid out of tissues and reduce recovery time;
9. Stimulate the flow of lymph, the body's natural defense system, against toxic invaders;
10. Release endorphins to control and relieve pain for chronic illness, injury and recovery from surgery;
11. Reduce post-surgery adhesions and edema and can be used to reduce and realign scar tissue after healing has occurred;
12. Improve neuromuscular efficiency;
13. Reduce trigger point sensitivity;
14. Optimize length-tension relationships throughout the human body which reduces stress on movement patterns.

Embodiments herein relate to therapeutic rollers, such as foam type rollers, that may include heat, cold and/or vibration producing elements such that the therapeutic roller may contribute multiple advantages to a user of such a roller above that of a standard foam roller.

Disclosed herein in various embodiments is a therapeutic roller, which may be a thermally active, or activatable, roller apparatus. In embodiments, the therapeutic roller may be any length between about 4 inches and about 36 inches in length with an overall outer diameter between about 2 inches and about 12 inches, although larger or smaller therapeutic rollers are contemplated. In some embodiments, the therapeutic roller may have a diameter between about 5 inches and about 8 inches. In some embodiments, the therapeutic roller may have a length between about 11 inches and 14 inches. The lengths and diameters described herein are intended as merely one set of examples, and in other embodiments the therapeutic roller may have a greater or smaller diameter and/or a greater or smaller length. The roller body may be a generally substantially cylindrical or a multi-faceted shape, for example, depending on the application. In some embodiments, the exterior surface of the therapeutic roller may be substantially smooth, while in others the exterior surface of the therapeutic roller may be textured, for example, regularly or irregularly textured, ribbed, faceted, or grooved and the like.

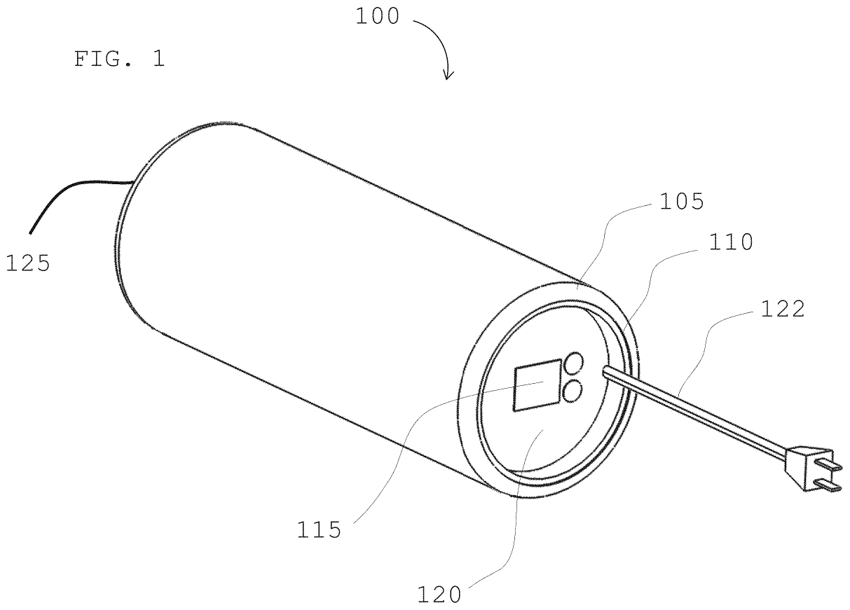

FIG. 1 depicts a thermally active roller apparatus 100, in accordance with various embodiments. The thermally active roller apparatus 100 includes a core 110, which may be hollow, for example, enclosing a cavity or compartment, or solid. The core may be formed from one or more parts. In some embodiments, the core 110 may be relatively hard or inflexible such that it provides support for the roller apparatus 100 and/or a person putting some or all of his or her body weight on the roller apparatus 100. For example, the core 110 may be constructed from plastic, metal, composites, wood, paper, rubber, or other structural material, for example, acrylonitrile butadiene styrene (ABS), polyvinyl chloride (PVC), or polycarbonate (PC).

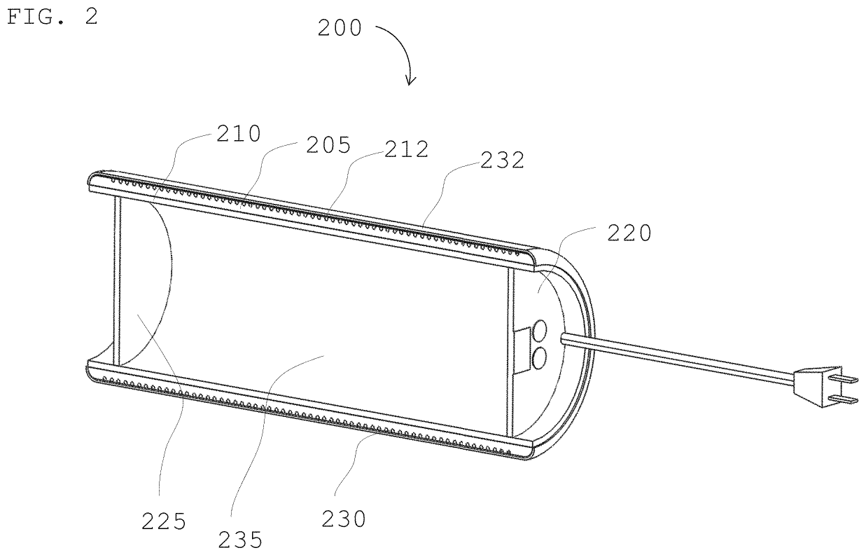

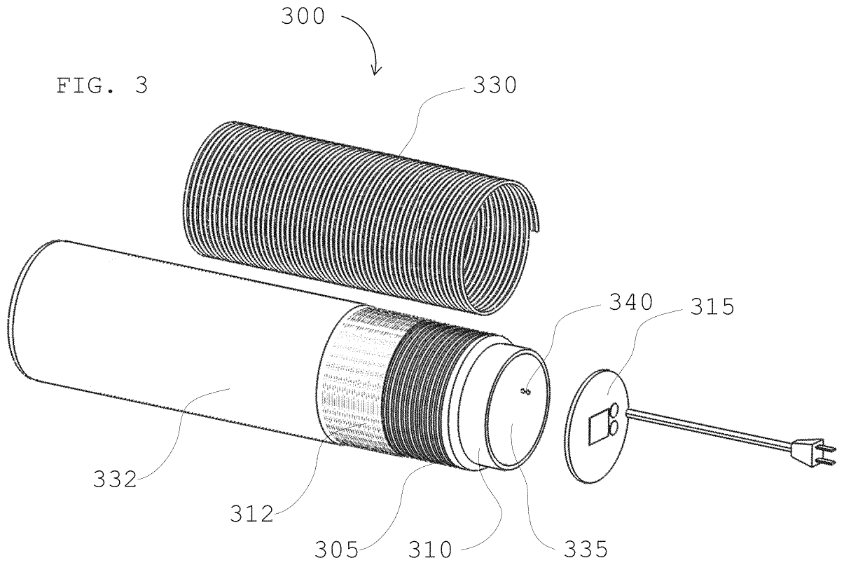

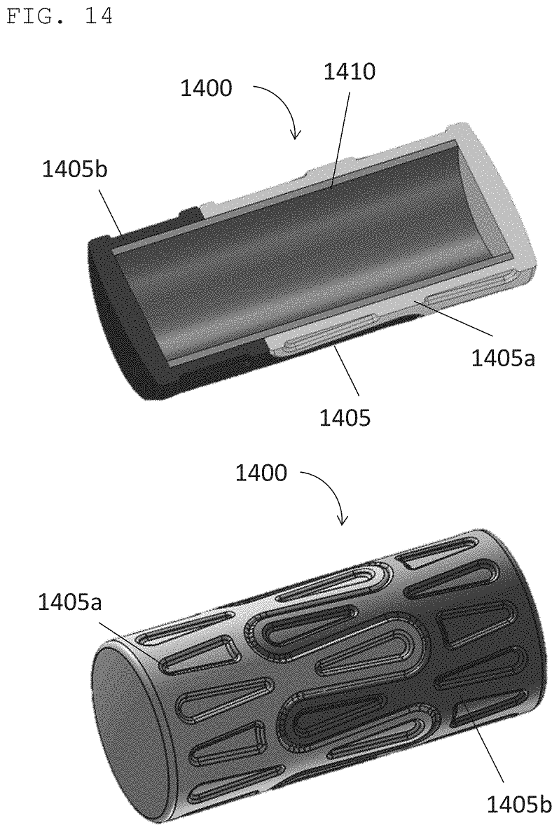

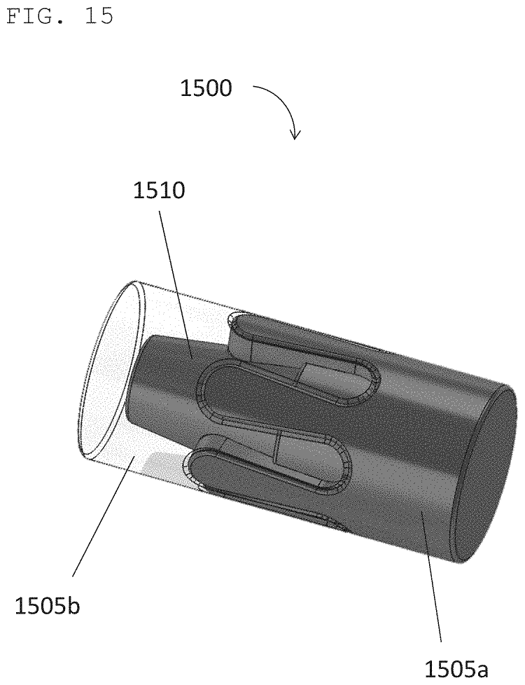

In the embodiment shown in FIG. 1, the roller apparatus 100 includes an endcap 120, although in certain embodiments, the endcap 120 may be omitted. The roller apparatus 100 may also have a second endcap 125, on the other end of the roller apparatus 100. As with the first endcap 120, the second endcap 125 may be omitted in certain embodiments. Thus, the roller apparatus 100 may have endcaps 120, 125 at or near one or both ends. The endcaps 120, 125 may be permanently fixed or removable to provide a storage compartment (see, for example, FIGS. 2 and 3, elements 235 and 335) in various embodiments. In certain embodiments, the core 110 may be hollow (as noted above), while in other embodiments the core may solid, for example, be replaced by a rigid cylinder, such as a foam cylinder. As shown in the depiction, the endcaps 120, 125 may include a cord 122, for supplying power and/or control electronics 115 for controlling the heating of the heated roller apparatus 100. In addition, a compliant layer 105, such as a comfort foam layer, may be included surrounding the core 110. In certain embodiments, such as shown in FIGS. 14 and 15, this compliant layer 105 may be composed of two or more elements having different (durometers) stiffnesses. In some embodiments, the compliant layer 105 may have a (durometer) stiffness value 10 Asker C to 65 Shore A, such as 25 Asker C to 60 Asker C.

FIG. 2 shows a cross-sectional diagram of the thermally active roller apparatus 200, in accordance with various embodiments. As shown, the core 210 may be covered in one or multiple layers, such as by a compliant layer 205, to improve comfort. The compliant layer 205 may include one or more materials such as open or closed cell foams such as polyurethane, EPP, EPS, EPE and EVA, foam rubbers, silicone rubber, thermoplastic elastomers, thermoplastic urethanes, textiles, or mechanical springs, with one or multiple (durometers) stiffnesses. In certain embodiments the thickness of the compliant layer 205 may vary from about 0.5 millimeters (mm) to about 74 mm. In some embodiments, the thickness of the compliant layer may vary from about 0.5 mm to about 12 mm. The compliant layer 205 may be permanently affixed to the core 210 or removable as part of the thermal system.

A layer of heating element or elements 230 may be placed on or near the outer surface of the compliant layer 205. The heating element 230 may take the form of resistive wire, resistive panels, heat pipes, heating coils or any thermoelectric heating apparatus. In various embodiments, the compliant layer 205 may have a variable surface thickness where the lower regions of the layer are shaped in such a way as to accept the heating elements 230, for example, ribbed and/or grooved. One example would be a series of helical grooves in the surface of the compliant layer 205 that may enable a thermal heating element 230, such as a wire, to nest in a fashion, for example, so that the combined material yield a consistent outer diameter. Heating elements 230 may be added over the entire length of the apparatus or over any fractional region thereof. Specifically, in some embodiments, heated regions may be interspaced with non-heated regions. In other embodiments, the spacing of heating elements 230 may be varied such that the amount of heat transferred varies over the length of the apparatus to create different temperature zones.

The device disclosed herein may have a heated region of between 80 and 140 square inches, ad more preferably 100 to 120 square inches. Resistive wire elements may generate approximately 40-160 watts and more preferably 70-90 watts. Power per unit may be approximately 0.40 to 0.90 watts per square inch. If the device generates too much heat, it will be unsafe. If it generates too little heat, the benefits of a heated foam roller will not be realized.

A thermally conductive layer 212 such as a layer of metal foil such as aluminum foil is placed over the heating elements 230. The layer is preferably thin and flexible and may be comprised of any material that readily conducts heat, such as metal foil. The thermally conductive layer may be bonded or mechanically affixed or merely tightly wrapped around the core or compliant layer. Preferably, it is placed in contact with or very close proximity to the heating elements 212.

Electrical connections between the control electronics and heating elements 230 may be isolated inside the core 210. A small passage or passages may be added to the core 210 and compliant layer 205 to allow electrical connection between the control electronics and the heating element(s) 230.

In order to ensure that the device does not become hot enough to cause injury, it may include means of governing temperature such that if the internal temperature of the device or the internal resistance of resistive wire heating elements exceeds a certain pre-determined threshold, then the power to the heating elements is reduced or turned off.

The thermally conductive layer disclosed herein conducts heat from the heating elements to the external surface of the device, as opposed to the core of the device. Existing heated rollers address concerns for user safety by providing an insulating layer between the heating elements and the external surface of the device, and commonly teach that such a layer is desirable or even necessary to ensure user safety. However, the inventor of the apparatus disclosed herein has discovered that when an insulating or non-conductive layer is positioned between the heating element and the surface of the device, the surface of the device does not remain hot enough for the benefits of the device to be obtained during repeated or prolonged use. When a heated roller is used to massage a human body, that human body absorbs heat and cools the surface of the device. The surface temperature of the device may decrease to a point where it is not hot enough to be satisfactory to the user, or to provide the benefits of heated massage. When this occurs, a user must suspend use so that the surface can re-heat. The conductive layer conducts heat preferentially to the surface of the device, and speeds re-heating of the surface of the device by conducting heat to the surface of the device.

When the device is equipped with a safety feature that reduces power to the device when the internal temperature of the roller or the internal resistance of the heating element exceeds a certain threshold, this feature will reduce ability of the device to recover surface temperature when it has been used. This is because the internal temperature of the device will remain high, and the device will reduce power to the heating element even though the surface has cooled. In this way, the safety feature interferes with the re-heating of the surface of the device. A thermally conductive layer that conducts heat to the surface helps resolve this problem.

A cover or comfort layer 232 may cover the conductive layer. This layer may be made of fabric, textile, or a film made from polymer, as discussed below.

Also shown in FIG. 2 the internal cavity or storage compartment 235 is located within the core 210 between the endcaps 220, 225. This cavity or storage compartment 235 may be used for the storage of components of the apparatus or other material. For example, the storage compartment 235 may be utilized to house electronic elements such as power cords, batteries and/or some other object; as storage for a thermal reservoir; or for items such as instruction manuals, comfort covers, or any range of items.

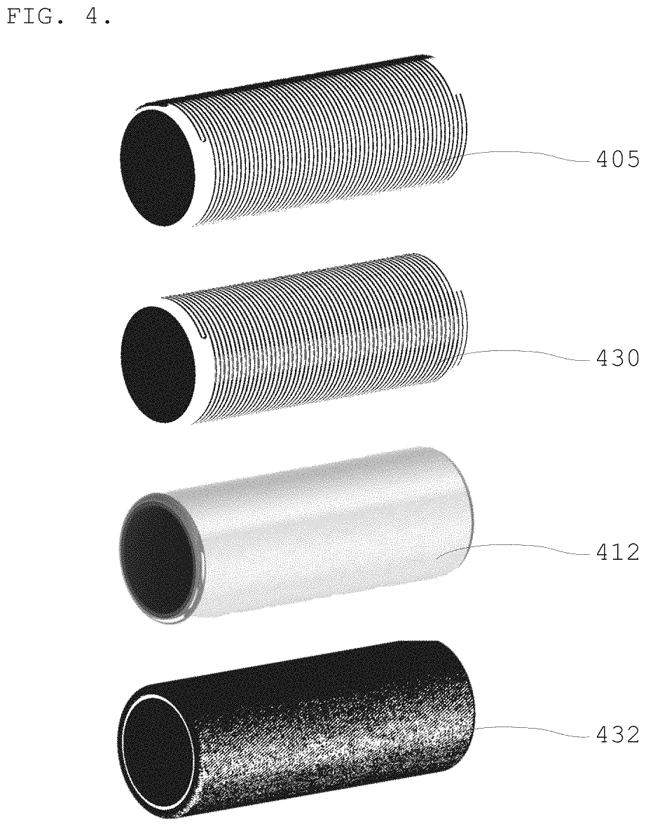

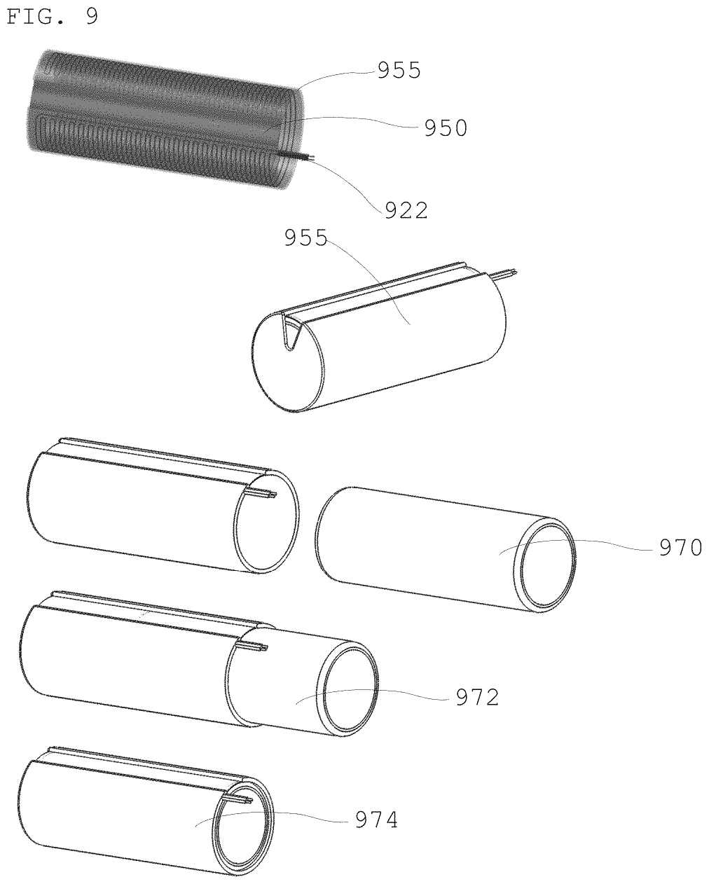

FIG. 3 depicts an exploded view of components of the thermally active roller apparatus 300, in accordance with various embodiments. FIG. 4 depicts individual layers of the roller apparatus, in accordance with various embodiments. FIG. 3 shows the core 310 and the compliant layer 305, which in this view includes grooves for placement of the heating elements 330. Electrical connections between the control electronics and heating elements 330 may be isolated inside the core 310. A small passage or passages 340 may be added to the core 310 and the compliant layer 305 to allow electrical connection between the control electronics 315 and the heating element(s) 330. Over the compliant layer 305 is the thermally conductive layer 312, which is in turn covered with an outer comfort layer 323, 332 or shell. For example, the roller body may be covered by a thin fabric or polymer cosmetic comfort layer 332 that may allow for easy heat transfer to the user. A few examples of such a cover or cosmetic/comfort layer 332 may include: natural or synthetic woven textiles and compliant films such as rubber or plastic. Comfort layer 332 may be permanently affixed by bonding directly to any of the substrate layers. In various embodiments the comfort layer 332 may be removable, for example, for cleaning or replacement. The comfort layer 332 may take the form of a flat or contoured fabric sheet that wraps around the apparatus and may be affixed via any temporary fastening means such as: hook and loop fasteners, buttons, snaps, zipper, laces or other. In other embodiments the comfort layer 332 may take the form of a sleeve or bag 955 into which the apparatus slides as depicted in FIG. 9. In the embodiment depicted in FIG. 9, the open end of the bag may have a closure system that allows electronics and power cables 922 to be tucked inside the bag for storage purposes. The bag may have a compliant region 950 that runs axially, relative to the roller. The compliant region may be created using elastic fabric or by other means and is intended to allow the bag to fit over a range of roller sizes. Elements 970, 972 and 974 show a roller cylinder being inserted into the bag embodiment. FIG. 4 separately shows the compliant layer 405, the heating elements 430, the thermal conductive layer 412, and the comfort layer or cover 432.

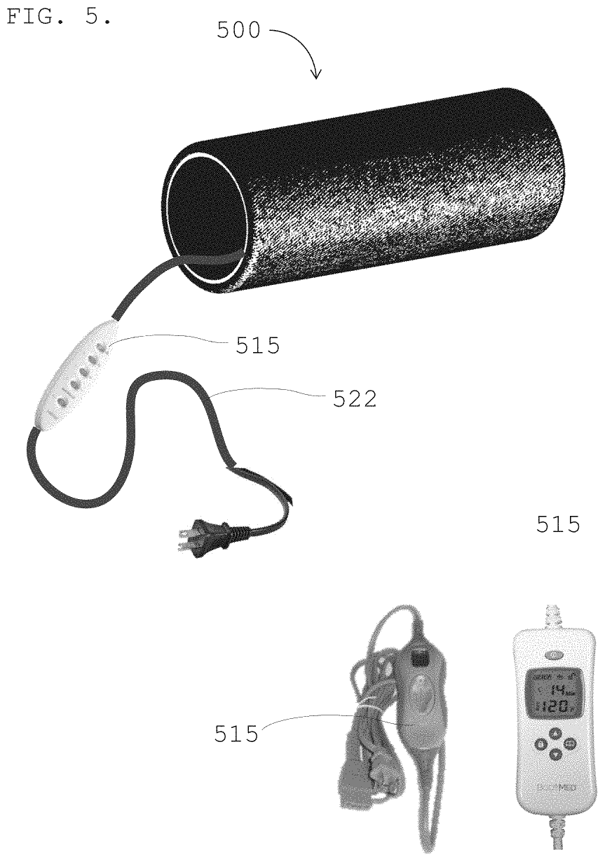

As depicted in FIG. 5, the electronic controls 515 for the apparatus may be located in any number of locations, such as on the body of the apparatus itself, on one or both endcaps for the body, along an AC power cord 522 as shown, at the wall plug or even controlled remotely by wireless remote or smartphone application. Control for the heater may take analog or digital form and may include any range of potential power settings ranging from a single or multiple digital settings to a continuous analog system utilizing rotary or linear potentiometer for infinite temperature control. Control systems may utilize an electronic graphical display to provide information on system status, power setting and actual temperature and/or include control electronics such as temperature controls and a timer. Controls may be any combination of buttons, sliders, switches, dials or touch sensitive technologies, LEDs, LCDs or other displays, for example, indicating time, temperature, power level and the like. In certain embodiments, electric power may be supplied by a standard AC power cord.

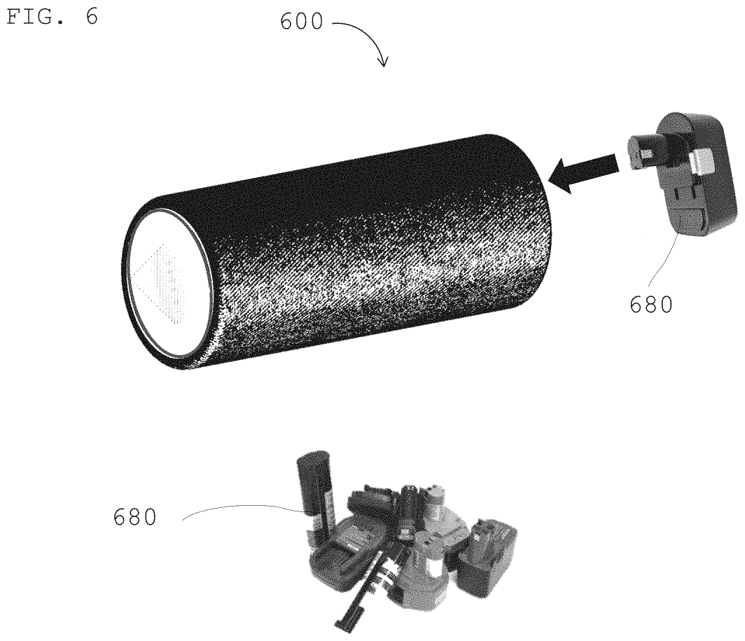

As shown in FIG. 6, in embodiments, power to the roller apparatus 600 may be supplied via rechargeable battery systems 680, which could be in addition to the AC power cord. Rechargeable batteries may be permanently affixed to the apparatus or removable for recharging. Removable batteries may be recharged via a charging station. Permanent batteries may be recharged via an AC power cord.

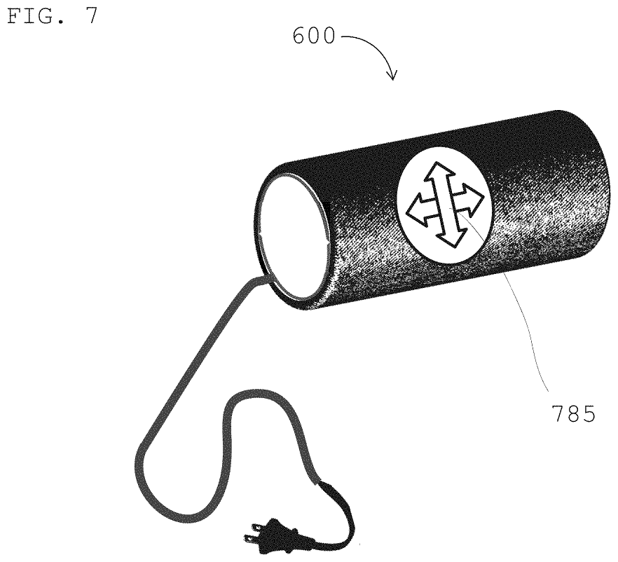

In other embodiments, such as those depicted in FIG. 7, components 785 of the roller apparatus 700 may be added to provide vibration to part or all of the apparatus. Vibratory motion may be supplied by any number of means. Some examples are AC or DC rotary motors with eccentric weights attached to the motor shaft, AC or DC rotary motors that drive a linearly oscillating mass, an electromagnetically driven oscillating mass, a pneumatically driven oscillation and/or a spring powered oscillating mass.

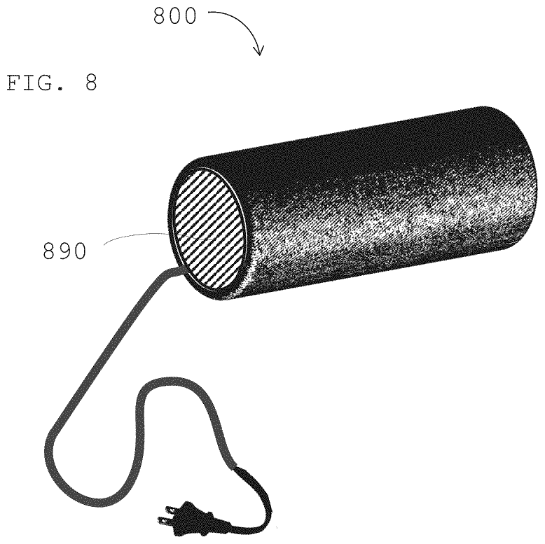

As shown in FIG. 8, additional embodiments of the disclosure may utilize a thermal reservoir 890, which may be located inside the core of the roller apparatus 800 to enable long periods of heating without power. The thermal reservoir 890 may include a solid and/or liquid mass with high specific heat capacity that may be conductively heated by the heating elements. Thermally conductive elements may then be used to transfer heat to the outer layer. In certain embodiments, hollow tubes may be used in place of conductive heating elements to circulate a liquid that would move heat from the reservoir to the outer layer.

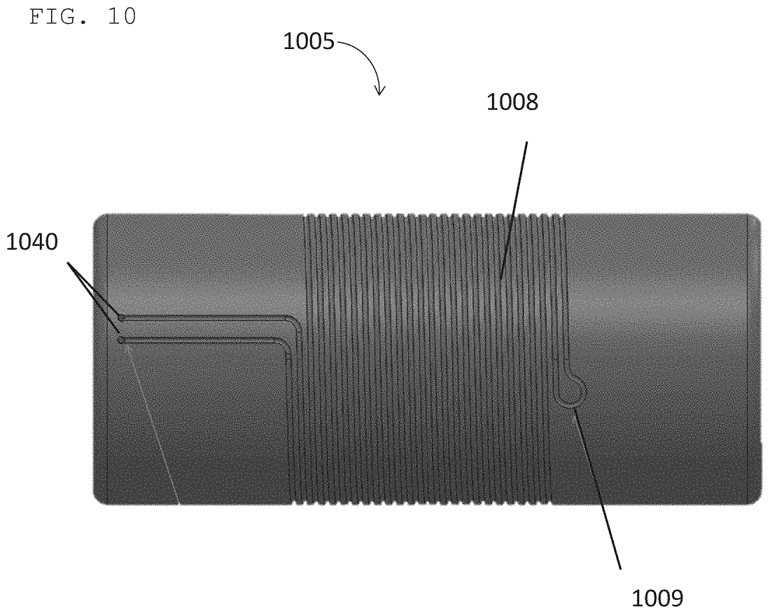

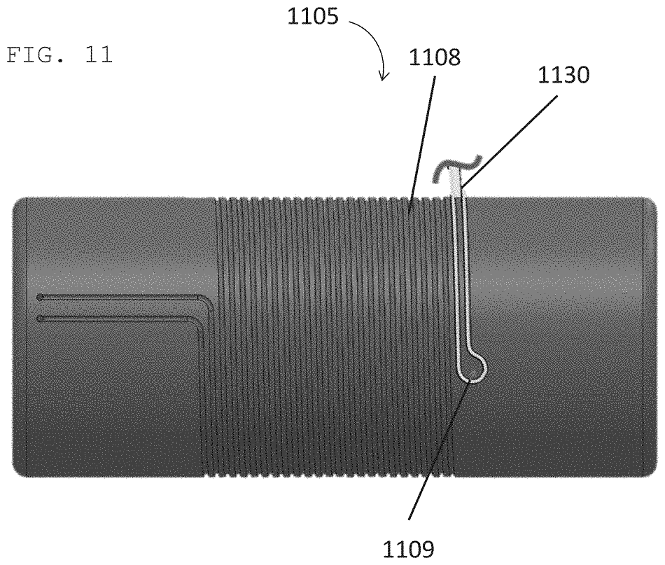

FIGS. 10 and 11 depict the loading of a heating element into the compliant layer 1005, in accordance with various embodiments. In the embodiment shown, twin helical grooves 1008 are molded or cut into the compliant layer 1005. A service loop 1009 may be included between the ends of the twin helical grooves 1008 to reduce stress on the heating element, such as a heating wire. In addition, holes 1040 in the compliant layer 1005 and the rigid core are present to allow wires to pass inside the roller apparatus to attach with the control electronics and power source. The heating element 1030 is folded in half (FIG. 11) and lays into the twin helical grooves 1008, starting at the service loop 1009 and then spiraling back to the controller end before passing through the holes 1006. After the heating element 1030 has been wound onto the compliant layer 1005 and secured, for example, via an adhesive or mechanical fastener, a large diameter thermally conductive layer may be used to capture the heating wire.

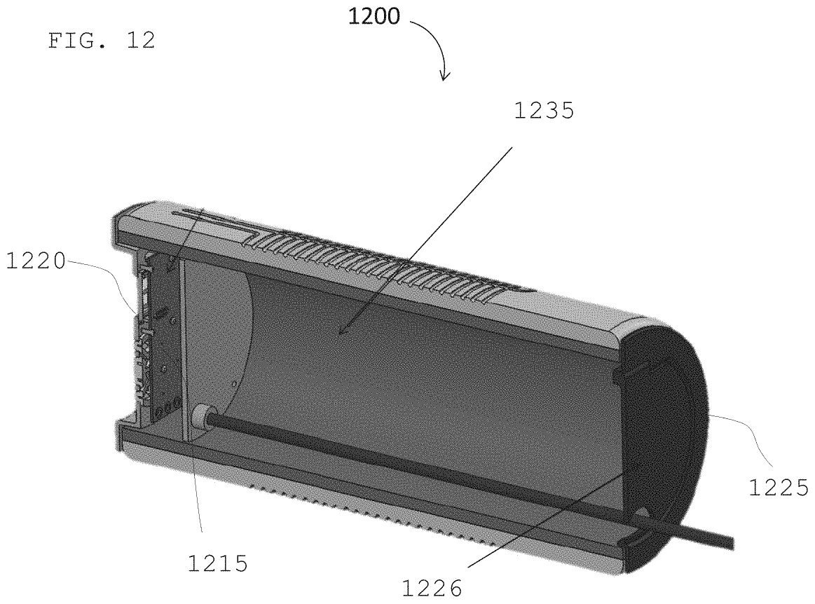

FIG. 12 depicts a cross-sectional view of a thermally active roller apparatus 1200 with comfort foam and heating elements, in accordance with various embodiments. The therapeutic roller 1200 shows the cavity 1235 bounded by endcaps 1220, 1225. Endcap 1220 is shown with control electronics 1215 and connected to the power cord 1222. The power cord 1222 passes out of the cavity 1235 through a hole in a door 1226 of the endcap 1225. In the embodiment shown, the electrical connections are made to a heating wire prior to endcap assembly into the end of the thermally active roller apparatus 1200 cylinder. The internal cavity 1235 can used for storage, for example, for power cord 1222, and accessed through the door 1226 in the endcap 1225.

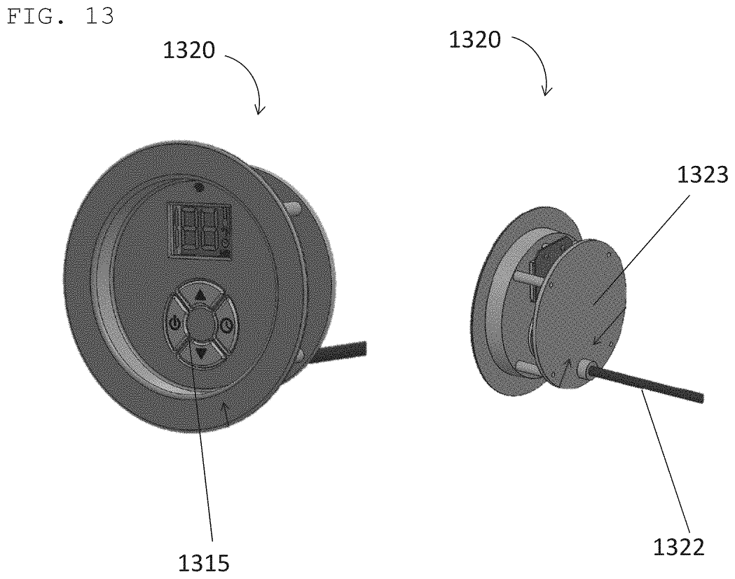

FIG. 13 depicts an endcap 1320, in accordance with various embodiments. The endcap 1320 may include control electronics 1315, such as temperature controls and a timer. The control electronics 1315 can include LEDs, such as an indicator light for power on. The control electronics 1315 can include LCDs or other displays, for example, indicating time, temperature, power level and the like. The back side of the endcap 1320 can include a connection for a power cord 1322, such as an endplate 1323 that reduces cord strain.

FIG. 14 depicts a therapeutic roller 1400, in accordance with various embodiments. In the embodiment shown, the core 1410 is covered by compliant layer 1405 that may be formed from two different portions 1405a, 1405b that have different stiffnesses. The two different portions 1405a, 1405b can be interdigitated about the center of the roller 1400, although it is possible that one portion may comprise more than half the roller 1400 surface and the other portion can cover the remainder. It is also contemplated that the compliant layer 1405 can be composed of more than two portions, each with different stiffnesses. Thus in certain embodiments the compliant layer may be formed or molded from two or more separate pieces of foam and assembled onto the core from either end. Using two or more interdigitated parts with different stiffnesses of the parts creates a much more dramatic “massage” feel, as well as providing multiple “regional” stiffness options. In other words, you could roll only on the soft end or only on the hard end or get the massaging feel of rolling right in the middle. In addition, the endcaps can be integral to the compliant layer 1405, which enables the benefits of a solid foam roller with the added stiffness of a rigid core roller. In some embodiments, a roller having two or more compliant layers of different stiffnesses includes heating. In some embodiments, a roller having two or more compliant layers of different stiffnesses does not include heating.

FIG. 15 depicts a roller 1500 having a compliant layer composed of two portions, and having a core 1510 that is integral, for example, formed together with, one of the two portions 1505a, 1505b of the compliant layer 1505. In some embodiments, the compliant layer is formed from two or more solid foam parts that overlap and interdigitate with no solid core. In some embodiments, a roller includes a solid core with any number of foam parts bonded to it, for example, having holes in the primary foam layer that are back filled with harder or softer “filler” pieces.

It will be apparent to those skilled in the art that various modifications and variations can be made in the disclosed embodiments of the disclosed device and associated methods without departing from the spirit or scope of the disclosure. Thus, it is intended that the present disclosure covers the modifications and variations of the embodiments disclosed above provided that the modifications and variations come within the scope of any claims and their equivalents.

1. A thermally active roller apparatus, comprising:
a core;
a compliant layer disposed about the core; a heating element disposed about the compliant layer, wherein the heating element comprises resistive wire, grooves are formed in the surface of the compliant layer, and the resistive wire is disposed within those grooves;
a thermally conductive layer disposed around compliant layer; and
a cover disposed about the compliant layer.

2. The thermally active roller apparatus of claim 1, wherein the core is hollow, forming a compartment.

3. The thermally active roller apparatus of claim 1, further comprising control electronics.

4. The thermally active roller apparatus of claim 1, further comprising a first endcap and, optionally, a second endcap, wherein the first endcap is disposed at a first end of the core.

5. The thermally active roller apparatus of claim 4, wherein the first endcap comprises electronic controls.

6. The thermally active roller apparatus of claim 1, further comprising a second endcap disposed at the second end of the core, wherein the second endcap comprises a door for accessing a compartment within the core.

7. The thermally active roller apparatus of claim 1, wherein the thermally conductive layer comprises metal foil.

8. The thermally active roller apparatus of claim 1, wherein the compliant layer comprises a pliable foam.

9. The thermally active roller apparatus of claim 1, wherein the grooves comprise twin helices.

10. The thermally active roller apparatus of claim 1, further comprising a power source comprising batteries, an interface for an external power source, or a combination thereof.

11. The thermally active roller apparatus of claim 1, further comprising a source of vibration.

12. The thermally active roller apparatus of claim 1, further comprising a power cord, which is retractable.

13. The thermally active roller apparatus of claim 1, wherein the compliant layer comprises two or more portions of different stiffnesses.

14. The thermally active roller apparatus of claim 13, wherein the two or more portions of different stiffnesses are interdigitated to lock the compliant layer in place about the core.

15. A roller apparatus, comprising a compliant layer comprising at least two portions of different stiffness and wherein the at least two portions are interdigitated to lock them together.
